(19) United States
(12) Patent Application Publication
(10) Pub. No.: US 2013/0041447 A1
(43) Pub. Date: Feb. 14, 2013

(54) DURABLE FINE WIRE LEAD FOR THERAPEUTIC ELECTROSTIMULATION AND SENSING

(71) Applicants: John L. Erb, Wayzata, MN (US); Kimberly Anderson, Eagan, MN (US); Paul A. Lovoi, Saratoga, CA (US)

(72) Inventors: John L. Erb, Wayzata, MN (US); Kimberly Anderson, Eagan, MN (US); Paul A. Lovoi, Saratoga, CA (US)

(73) Assignee: CARDIA ACCESS, INC., Eden Prairie, MN (US)

(21) Appl. No.: 13/648,840

(22) Filed: Oct. 10, 2012

Related U.S. Application Data
(63) Continuation of application No. 12/932,782, filed on Mar. 4, 2011, now abandoned.
Application No. 12/932,782 is a continuation-in-part of application No. 12/156,129, filed on May 28, 2008, now abandoned.
Application No. 12/156,129 is a continuation-in-part of application No. 12/584,837, filed on Sep. 10, 2009.
Application No. 12/932,782 is a continuation-in-part of application No. 12/590,851, filed on Nov. 12, 2009.
(60) Provisional application No. 61/191,722, filed on Sep. 10, 2008.
(60) Provisional application No. 61/198,900, filed on Nov. 12, 2008.

(51) Int. Cl. A61N 1/05; B05D 5/12; H01R 43/16

(52) U.S. Cl. 607/119; 29/874; 427/2.24

(57) ABSTRACT

A cardiac pacemaker, other CRT device or neurostimulator has one or more fine wire leads. Formed of a glass, silica, sapphire or crystalline quartz fiber with a metal buffer cladding, a unipolar lead can have an outer diameter as small as about 300 microns or even smaller. The buffered fibers are extremely durable, can be bent through small radii and will not fatigue even from millions of iterations of flexing. Bipolar leads can include several conductors side by side within a glass/silica fiber, or can be concentric metal coatings in a structure including several fiber layers. An outer protective sheath of a flexible polymer material can be included.

### RELATED PATENT DOCUMENTS

This application is a continuation of U.S. patent application Ser. No. 12/932,782 filed Mar. 4, 2011; which is a continuation-in-part of U.S. patent applications, Ser. No. 12/156,129 (ABN) filed May 28, 2008, Ser. No. 12/584,837 filed Sep. 10, 2009 (which claimed benefit from provisional application Ser. No. 61/191,722 filed Sep. 10, 2008) and Ser. No. 12/590,851 filed Nov. 12, 2009 (which claimed benefit from provisional application Ser. No. 61/198,900 filed Nov. 12, 2008). This application incorporates by reference in its entirety U.S. patent application Ser. No. 12/584,837 filed Sep. 10, 2009 (U.S. Pub. No. 2010/0331941). This application also incorporates by reference in its entirety U.S. patent application Ser. No. 12/586,031 (U.S. Pub. No. 2010/0057179 published Mar. 4, 2010). In addition, the following unpublished applications are incorporated entirely herein: Ser. No. 12/590,851 filed Nov. 12, 2009, and Ser. No. 12/660,344 filed Feb. 23, 2010.

### BACKGROUND OF THE INVENTION

This invention concerns wiring for electrostimulation and sensing devices such as cardiac pacemakers, ICD and CRT devices, and neurostimulation devices, and in particular encompasses an improved implantable fine wire lead for such devices, a lead of very small diameter and capable of repeated cycles of bending without fatigue or failure. The term therapeutic electrostimulation device (or similar) as used herein is intended to refer to all such implantable stimulation and/or sensing devices that employ wire leads.

Pacing has become a well-tested and effective means of maintaining heart function for patients with various heart conditions. Generally pacing is done from a control unit placed under but near the skin surface for access and communications with the physician controller when needed. Leads are routed from the controller to the heart probes to provide power for pacing and data from the probes to the controller. Probes are generally routed into the heart through the right, low pressure, side of the heart. No left, high pressure, heart access through the heart wall has been successful. For access to the left side of the heart, lead wires are generally routed from the right side of the heart through the coronary sinus and into veins draining the left side of the heart. This access path has several drawbacks; the placement of the probes is limited to areas covered by veins, leads occlude a significant fraction of the vein cross section and the number of probes is limited to 1 or 2.

Over 650,000 pacemakers are implanted in patients annually worldwide, including over 280,000 in the United States. Over 3.5 million people in the developed world have implanted pacemakers. Another approximately 900,000 have an ICD or CRT device. The pacemakers involve an average of about 1.4 implanted conductive leads, and the ICD and CRT devices use on average about 2.5 leads. These leads are necessarily implanted through tortuous pathways in the hostile environment of the human body. They are subjected to repeated flexing due to beating of the heart and the muscular movements associated with that beating, and also due to other movements in the upper body of the patient, movements that involve the pathway from the pacemaker to the heart. This can subject the implanted leads, at a series of points along their length, through tens of millions of iterations per year of flexing and unflexing, hundreds of millions over a desired lead lifetime. Previously available wire leads have not withstood these repeated flexings over long periods of time, and many have experienced failure due to the fatigue of repeated bending.

Neurostimulation refers to a therapy in which low voltage electrical stimulation is delivered to the spinal cord or targeted peripheral nerve in order to block neurosensation. Neurostimulation has application for numerous debilitating conditions, including treatment-resistant depression, epilepsy, gastroparesis, hearing loss, incontinence, chronic, untreatable pain, Parkinson's disease, essential tremor and dystonia. Other applications where neurostimulation holds promise include Alzheimer's disease, blindness, chronic migraines, morbid obesity, obsessive-compulsive disorder, paralysis, sleep apnea, stroke, and severe tinnitus.

Today's pacing leads manufactured by St. Jude, Medtronic, and Boston Scientific are typically referred to as multifilar, consisting of two or more wire coils that are wound in parallel together around a central axis in a spiral manner. This construction technique helps to reduce impedance in the conductor, and builds redundancy into the lead in case of breakage. The filar winding changes the overall stress vector in the conductor body from a bending stress in a straight wire to a torsion stress in a curved cylindrical wire perpendicular to lead axis. A straight wire can be put in overall tension, leading to fatigue failure, whereas a filar wound cannot. However, the bulk of the wire and the need to coil or twist the wires to reduce stress, limit the ability to produce smaller diameter leads.

Modern day pacemakers are capable of responding to changes in physical exertion level of patients. To accomplish this, artificial sensors are implanted which enable a feedback loop for adjusting pacemaker stimulation algorithms. As a result of these sensors, improved exertional tolerance can be achieved. Generally, sensors transmit signals through an electrical conductor which also serve as the pacemaker lead that enables cardiac electrostimulation. In fact, the pacemaker electrodes can serve the dual functions of stimulation and sensing.

It is the object of the invention described herein to overcome the problems of previously available implantable leads for electrostimulation and sensing, including pacemakers, ICD and CRT devices, and neurostimulation devices, with leads which are small in diameter and will exhibit very long-term durability.

### SUMMARY OF THE INVENTION

In the invention a flexible and durable fine wire lead for implanting in the body, connected to a pacemaker, ICD, CRT or other cardiac pulse generator, is formed from a drawn silica, glass, sapphire or crystalline quartz fiber core with a conductive metal buffer cladding on the core. There can additionally be a polymer coating over the metal buffer cladding, which may be biocompatible and resistant to environmental stress cracking or other mechanism of degradation associated with exposure and flexure within a biological system. The outer diameter of the fine wire lead preferably is less than about 750 microns, and may be 200 microns or even as small as 50 microns. Metals employed in the buffer can include aluminum, gold, platinum, titanium, tantalum, or others, as well as metal alloys of which MP35N, a nickel-cobalt based alloy, is one example. In a preferred embodiment the metal cladding is aluminum or gold, applied to the drawn silica, glass, sapphire or crystalline quartz fiber core immediately upon drawing and providing a protective hermetic seal over the fiber core.

If more than one conductor is needed, multiple unipole fibers can be used with one conductor per fiber. However, in another alternative the silica or other type fiber is used as a dielectric with a wire in the center of the fiber core as one conductor and the metallic buffer layer on the outside of the fiber core, both protecting the fiber and acting as the coaxial second conductor or ground return.

In a third embodiment, a further layer of silica, glass, etc. (as above) covers the metallic cladding, with a further electrically conductive buffer covering that dielectric layer. This embodiment may be with or without a center wire in the inner fiber. These silica, glass, etc. layers and buffer coatings can be continued for several more layers to produce a multiple conductor cable.

In a fourth embodiment the center of the fiber core is hollow to increase flexibility of a lead of a given diameter. In a fifth embodiment multiple conductors are embedded side-by-side in the silica, glass, etc. fiber core.

A further embodiment has the conductive lead composed of many smaller metal-buffered or metal wire-centered silica or glass fibers that together provide the electrical connection. This embodiment allows for high redundancy for each connection and very high flexibility.

The fiber coax is extremely strong and flexible. The current requirement for pacemaker leads does not dictate large central conductors, so that a few mils are sufficient (about 25 to 50 microns or so). The voltage used is very low, so insulator thickness requirements are minimal. The invention contemplates cables (meaning fiber-core leads with one or more conductors) of 100 to 200 micron diameter, and even unipolar cables as small as 50 microns in diameter or even smaller. These cables will have the flexibility to provide delivery to any portion of the heart.

Another advantage of using a coax fabricated from a silica/glass fiber insulator is that the connection between the cable and pacing probe (or a hub as discussed below) can be hermetic and therefore robust. Any portion of the fiber that is not protected from water or water vapor, such as in normal atmosphere, will rapidly degrade in strength due to the formation of surface cracks. This will allow that portion of the fiber to lose significant strength. Hermetically sealing the processed ends of the fiber cable will ensure that it remains rigid and protected, thus preserving the very high strength and fatigue resistance of the flexible portion of the fiber cable. Hermetic sealing is enabled by the use of an inorganic, high-temperature dielectric, glass or silica, which can be fused together with a similar dielectric, which is not the case with leads with organic materials. Hermeticity can be achieved whether the device is in the form of a coax or individual fibers cabled together, as long as an impervious surface seal is applied. This sealed approach can also be used with industry standard conductors such as an IS-1 making the lead compatible with most manufacturers' pacing products.

The fiber coax is a combination of technologies that have been developed for different applications. Optical fiber cable is produced from a draw tower, a furnace that melts the silica or glass (or grown crystals for sapphire or quartz) and allows the fiber to be pulled, “drawn”, vertically from the bottom of the furnace. Fibers produced in this manner have strength of over 1 Mpsi. If the drawn fiber is allowed to sit in normal atmospheric conditions for more than a few minutes, that strength will rapidly be reduced to the order of 2-10 kpsi. This reduction is caused by water vapor attack on the outer silica or glass surface, causing minute cracking. Bending the fiber causes the outside of the bend to be put into tension and the cracks to propagate across the fiber causing failure. To ensure that the fiber remains at its maximum strength, a buffer is added to fibers as they are drawn. As the fiber is drawn and cools, a plastic coating, the buffer, is applied in a continuous manner protecting the fiber within a second of being produced.

The TOW missile was developed during the 1960s as an antitank missile for the U.S. Army. The missile was launched from a shoulder mounted launcher and was guided to the target by an optical system that included a fiber spooled from the rear of the missile as it flew. The fiber had to be very strong and light to unreel several kilometers of fiber in a few seconds. Fiber optics were selected but to further strengthen the fiber and protect it from damage, the plastic buffer was replaced with a metal buffer. The metal buffer used at that time was aluminum, but systems to coat fibers with gold and other metals have since been developed.

The patents for the metal buffer technology covered a wide range of metals and alloys and were issued to Hughes in 1983 (U.S. Pat. Nos. 4,407,561 and U.S. 4,418,984).

The concept of using the fiber optical systems as a coax was developed for micro miniature x-ray sources by Xoft, Inc., Photoelectron Corporation and others. See U.S. Pat. Nos. 6,319,188 and 6,195,411. These fibers were used because they provided high flexibility, high voltage hold-off and direct connection to the x-ray source without a joint between the x-ray source and the HV power supply. The standard available optical fiber did not include a central electrical conductor. To include a wire in the center of the fiber, the wire must be drawn with the silica, glass, etc. fiber in the draw tower. For optical applications, to ensure that any optical energy launched into the fiber is not absorbed at the core wire interface, an additional lower index silica or glass cladding is provided between the core and the wire. All this is known prior practice.

Alternative methods of producing fiber coax include drawing a core fiber, coating that core with a metal buffer and drawing additional silica or glass over the assembly and cladding that final assembly with an additional metal buffer. Fibers can be pulled with a hole in the center as well, increasing flexibility; hole diameter can vary. In one embodiment one or more wires can be put inside the hole through a fiber. The fiber can be redrawn to engage the wire if desired.

Additional embodiments can also be used where the fiber, either solid core or hollow, can act as the strength member and dual electrical conductors can be placed outside the fiber system and separated by plastic or polymer insulators. Fatigue of metals and plastics after millions of small deflection stresses is one of the life-limiting aspects of conventional pacing leads. Silica, glass, etc. fibers protected with robust buffer systems will not exhibit fatigue. Fatigue in silica or glass is caused by propagation of cracks, which are present at low levels in typical silica or glass fibers as produced for standard communication purposes. Typically they exhibit only a few surface flaws per kilometer of fiber. Therefore silica or glass fiber coax cables make ideal pacing leads: small diameter, low mass, highly flexible, robust and with very long service life.

One method according to the invention for testing fibers for leads is to stretch a long segment until it breaks; the weakest point in the fiber will break first. If the fiber meets some minimal standard for tensile strength, then the entire fiber meets that strength minimum and flaws will not exist up to some level. If the fiber does break, the remaining pieces can be similarly tested. As this is repeated the limits at which the fiber will break will continue to climb, allowing selection of extremely flaw-free sections of fiber. This will further enhance the ability of the fiber to resist failure due to repeated stress cycling. This is a type of fiber “proofing”, but proofing as previously known was for lot testing rather than for selections of sections of highest strength from a fiber. Pursuant to the invention fibers for use in the fine wire leads are proofed to at least about 90% of the intrinsic strength value of the material, or more broadly, at least about 75%.

The invention also contemplates a pacing system that avoids problems encountered in prior systems and provides more versatility than previously available. In this system a control hub is implanted in the pericardia region of the heart. The hub is connected to each pacing site by a silica, glass, etc. cable conductive lead of one of the types described above. The hub in turn is connected to the pacemaker by a single lead using the same technology. The hub system provides for shorter, localized connections involving a plurality of conductive leads, often five or six or more, while only one lead need be implanted between the hub and the pacemaker, normally implanted higher in the chest and just under the skin. The hub or pacing can could also serve as a depot for electronics that would process sensing information received from electrodes attached distally to the hub, in order to select which leads receive stimulation. This has implications for situations in which a first lead shows signs of dysfunction; the electronics can be switched by the physician to stimulate a different lead. Alternatively, if the desired location of stimulation changes chronically due to physiological changes in the heart, electronics can sense this and provide the physician with information needed to enable selection of a different lead for stimulation.

The glass/silica fine wire lead of the invention is compatible with drug/steroid elution for controlling fibrosis adjacent to the lead electrode, which is a known technique used with conventional pacing leads for controlling impedance and thus battery life. For example a bioerodable polymer can be positioned on the distal end of a lead at the electrode, the polymer containing the eluting drug.

The fine wire leads of the invention can employ anchoring systems for stabilizing the fiber lead against unwanted migration within the coronary vein. Such anchoring systems can consist of expandable/retractable stents attached to the lead, or helical, wavy, angled, corkscrew, J-hook or expandable loop-type extensions attached to the lead, that take on the desired anchoring shape after delivery of the lead from within a delivery catheter.

Delivery devices can be used for installation of the fine wire leads of the invention. A steerable catheter for example, can be used and then removed when the leads are properly deployed in the proper anatomical positions.

It is among the objects of the invention to improve the durability, lifetime flexibility and versatility of wire leads for pacemakers, ICDs, CRTs and other cardiac pulse generators, as well as electrostimulation or sensing leads for other therapeutic purposes within the body. These and other objects, advantages and features of the invention will be apparent from the following description of preferred embodiments, considered along with the accompanying drawings.

### DESCRIPTION OF THE DRAWINGS

FIG. 1 is a schematic view partially cut away, showing a human heart and indicating a path of pacemaker or other cardiac pulse leads in accordance with conventional practice.

FIG. 2 is a schematic drawing in perspective showing one embodiment of an implantable fine wire lead for a cardiac pulse generator such as a pacemaker.

FIG. 3 is a similar view showing another embodiment of a fine wire lead.

FIG. 4 is a view showing a further embodiment of a fine wire lead.

FIG. 5 is a view showing another embodiment of a fine wire lead.

FIG. 6 is a view showing an embodiment with twisted or braided multiple conductors.

FIG. 7 is a schematic perspective view showing another form of fine wire pacing lead.

FIG. 8 is a sectional view showing a connector at an end of a lead of the invention.

### DESCRIPTION OF PREFERRED EMBODIMENTS

The invention encompasses all implantable electrostimulation devices with implanted wire leads, but is illustrated in the context of a cardiac pulsing device. Typically, a pacemaker is implanted just under the skin and on the left side of the chest, near the shoulder. The heart is protected beneath the ribs, and the pacemaker leads follow a somewhat tortuous path from the pacemaker under theclavicle and along the ribs down to the heart.

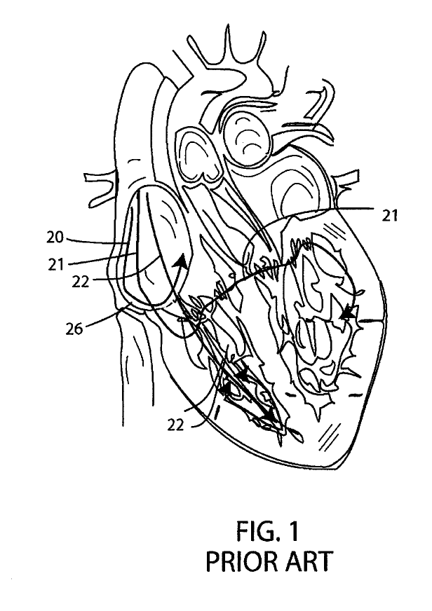

FIG. 1 shows schematically a human heart with some walls cut away. In FIG. 1 pacing leads are shown following a conventional path into the heart, and into the cardiac veins of the left ventricle, as has been typical of conventional practice and which, with some exceptions, is the basic path of leads of this invention.

In typical conventional practice, conductive leads 20, 21 and 22 are introduced into the heart through the superior vena cava 24, brought into the vena cava via subclavian or cephalic vein access points. For the right side of the heart, separate conventional pacing electrodes, as well as separate electrodes for biventricular pacing are normally routed into right ventricle, as well as the right atrium. For the left ventricle, typically a wire lead 21 would be brought from the right atrium 26 into the coronary sinus, and from there the leads are extended out into one or more coronary veins adjacent to the surface of the left side of the heart. The leads are not introduced directly into the interior of the left ventricle, which is the high pressure chamber.

Pursuant to the invention the routing of silica/glass fiber leads can be essentially the same as with conventional leads. An important difference is that the silica/glass lead, being much smaller diameter than conventional leads, can be positioned deeper and more distally (also “retrograde” to normal blood flow toward the coronary sinus) within the target coronary vein. The coronary sinus/coronary vein architecture can be a relatively tortuous path, such that the physician will have an easier time manipulating a smaller diameter, flexible lead into the desired position within the coronary vein than for a larger diameter lead. Also, as a lead is manipulated deeper (more distally) within the coronary vein, the diameter of the vein becomes progressively narrowed. Thus, a smaller diameter lead can be placed deeper than a larger diameter lead. One theoretical reason why it is useful to place the terminal electrode of the lead in the deeper/distal/narrower portion of the coronary vein is that that portion of the vein apparently lies closer to myocardium. Thus, the cardiac muscle can perhaps be stimulated with less energy use when the electrode is closer to intimate contact with muscle overlying the coronary vein.

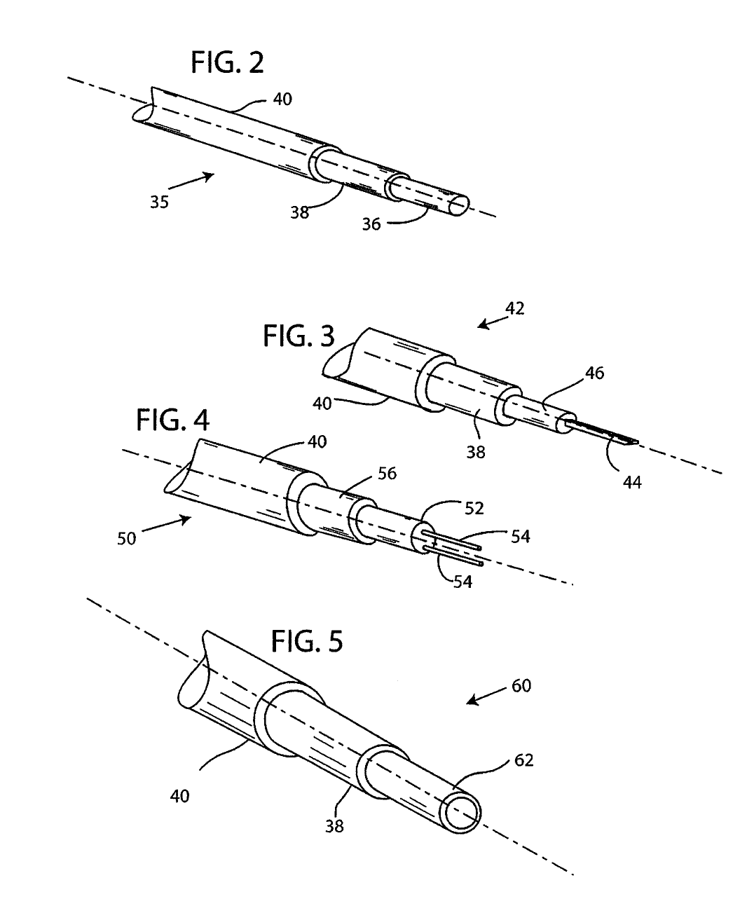

FIG. 2 is a simple schematic showing one preferred embodiment of an implantable fine wire lead 35 pursuant to the invention, for subdermal connections from a pulsing device to the heart. In this form the lead 35 is unipolar. It has a drawn fiber core 36 of glass, silica, sapphire or crystalline quartz (“glass/silica” or “silica/glass”) with a conductive metal buffer 38 over the fiber core. As discussed above, the buffer 38 is coated onto the fiber immediately upon drawing of the fiber, to preserve the strength of the fiber, protecting it from environmental elements such as atmospheric moisture that can attack the glass/silica surface and introduce fine cracking. Aluminum is a preferred metal buffer 38 because of its hermetic bonding with the silica or glass surface, although gold or other suitable metals or metal alloys can be used. The aluminum buffer can be about 20 microns thick, or 5 microns thick or even thinner. The wire lead 35 will have an electrode (not shown) at its distal end.

FIG. 2 also shows a polymer coating 40 as an outer buffer. This buffer is also added very soon after drawing, and is applied after the metal buffer 38 in a continuous manner. The plastic outer buffer coating 40 is biocompatible. As discussed further below, a further metal buffer can be added over the aluminum buffer 38 prior to addition of the plastic coating. This can be a coating of gold or platinum, for example, both of which are biocompatible. The plastic buffer 40 adds a further protective layer.

FIG. 3 shows a modified fine wire pacing lead 42 which has a metal conductor 44 as a center element. Here, the pure silica/glass fiber core 46 is drawn over the metal conductor 44. The process is well known, with a hollow glass/silica fiber first produced, then a metal conductive wire placed through the hole in the fiber and the glass/silica fiber drawn down against the wire. A conductive metal buffer is shown at 38 over the fiber, having been applied immediately on drawing of the conductor-containing fiber 46. An outer buffer coating of polymer material is shown at 40, being biocompatible and serving the purposes described above.

FIG. 4 is a similar view, but in this case showing a fine wire lead 50 formed of a glass/silica fiber core 52 formed over two metal conductors 54. The wire is precoated with a thin layer of glass before being co-drawn with fiber. An aluminum buffer coating 56 surrounds the silica fiber 52, protecting the fiber from deterioration as noted above, and this can serve as a third conductive lead if desired. Again, an outer polymer buffer 40 provides an outer protective jacket and is biocompatible.

In FIG. 5 is shown another embodiment of a fine wire pacing lead 60 of the invention. In this case the glass/silica fiber core 62 is hollow, allowing for better flexibility of the lead, and the lead construction is otherwise similar to that of FIG. 2.

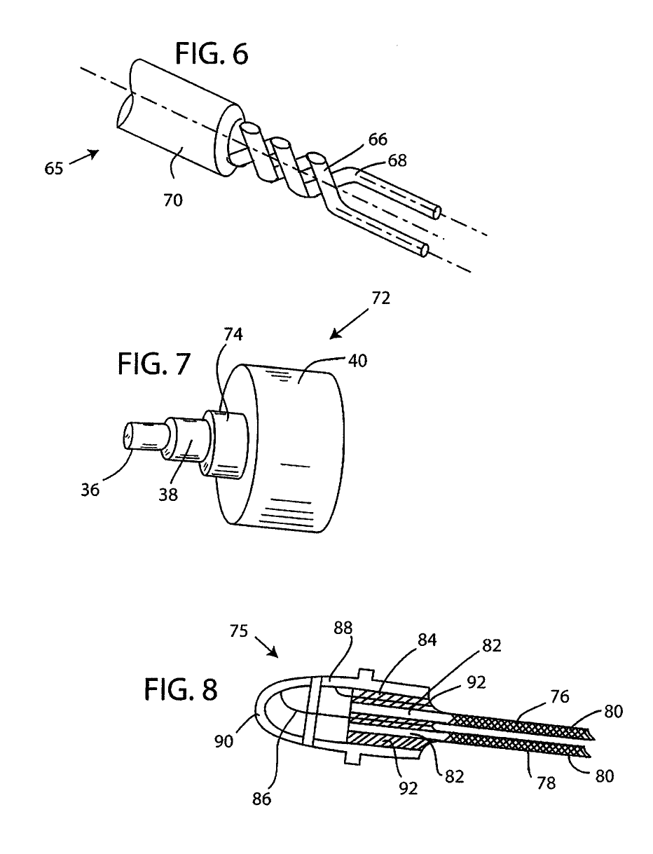

FIG. 6 shows a modified embodiment of a fine wire pacing lead 65 which has multiple glass/silica fibers 66 and 68 in a helical interengagement, twisted together. Each lead 66, 68 comprises a glass/silica fiber conductor which can be similar to what is shown in FIG. 2, with or without a polymer buffer coating 40, or each could be constructed in a manner similar to FIG. 3, with or without a plastic buffer coating. Although two such fiber leads are shown, three or more could be included. The glass/silica fiber cores provide for strength and small-radius bending of the helical leads 66, 68, and this type of braiding or helical twisted arrangement is known in the field of pacing leads, for absorbing stretching, compression or bending in a flexible manner An outer polymer coating 70 protects the assembled fiber leads and provides biocompatability. The leads 66, 68 themselves can have the aluminum or other metal cladding as their outer layer, or they can have a further cladding of biocompatible metal or polymer.

FIG. 7 shows a section of a fine wire lead 72 which is similar to that of FIG. 2, with a silica core 36 and an aluminum cladding 38, but with a further biocompatible metal cladding 74 over the aluminum cladding. As noted above, this can be gold or platinum, for example. The outer layer of polymer material is shown at 40.

FIG. 8 shows a terminal or connector 75 of the invention, formed at the end of two silica/glass fiber conductors 76 and 78 each of which may be formed as described above, with a conductive buffer 80 on the exterior of each. In the type of connector 75 shown in FIG. 8, the glass/silica fibers 82 of each of the separate leads 76 and 78 extend into the connector as shown. A high temperature wire 84, 86 is welded to each of the conductive buffer claddings 80 of the two leads 76 and 78, respectively. This welded connection is made essentially outside the terminal 75, to the right as viewed in FIG. 8, where the cladding 80 on the fibers will not be oxidized or rendered non-conductive by the formation of the terminal. These wires, preferably of Kovar, are connected to respective ones of two electrically isolated sections 88 and 90 of the terminal. The two sections 88 and 90 are of conductive metal and are adapted to plug into a socket formed to receive this connector 75.

Inside the connector 75, the fibers and conductive wires 84, 86 are sealed within the connector portion 88 using a relatively low temperature glass 92. The connector wires 84, 86, if of material such as Kovar, will not deteriorate even if a high temperature glass is used for sealing. The glass seal 92 does not extend over the weld connection from the wires 84, 86 to the buffer 80 on each of the leads 76 and 78. These weld connections and the unprotected portions of the wires 84, 86 need to be protected, covered by an appropriate material at the back end of the connector 75, where the two leads 76 and 78 emerge from the connector. They can be covered by a polymer, or more preferably a metal buffer can be applied to each individual wire/buffer 80 connection. This could be done before or after sealing with the glass seal 92. If a high temperature transition metal such as platinum is used for this purpose, the connection between the Kovar wire and the fiber could be protected from a high temperature glass seal 92, assuming a high temperature material is used here, in the case where the glass seal 92 is applied after the Kovar wire connection is made to the fiber. In this way a hermetic seal is achieved, and analogous connectors can be formed on unipolar, single-fiber leads or on bipolar leads having an exterior buffer and an interior wire.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

What is claimed is:

1. A flexible microthin fine wire or lead suitable for in vivo implantation, comprising a dual layer thin metal conductive buffer cladding on a nonconducting core.

2. The flexible wire or lead of claim 1, wherein the nonconducting core includes polymer, glass, silicon, ceramic or combinations thereof.

3. The flexible wire or lead of claim 1, wherein the dual layer thin metal conductive buffer cladding is aluminum, titanium, platinum gold, silver, or combinations thereof.

4. The flexible wire or lead of claim 1, wherein the dual layer thin metal conductive buffer cladding includes a first deposited layer of metal particulates having a size less than 1 micron.

5. The flexible wire or lead of claim 4, wherein the dual layer thin metal conductive buffer cladding includes a second deposited layer of metal macroparticles having a size greater than 1 micron.

6. The flexible wire or lead of claim 1, wherein the wire has a flexibility of up to five million cycles.

7. The wire or lead of claim 1 wherein the wire or lead includes a polymer.

8. The wire or lead of claim 7 wherein the wire or lead has a diameter of about 150 to about 250 nm.

9. The wire or lead of claim 1, including a cardiac pacemaker lead.

10. The wire or lead of claim 1, wherein the lead is configured and arranged for attachment to a device for controlling or monitoring electrical input.

11. A method of making a flexible fine wire or lead, the method comprising the steps of:
providing a non-conducting core made of glass, or silica or combinations thereof,
forming a first metal layer on the core by deposition of aluminum, titanium, platinum, gold, silver or combinations thereof, and
depositing a second metal layer on the first metal layer.

12. The method of claim 11, wherein the step of depositing the second metal layer includes ion plasma deposition.

13. The method of claim 11, wherein the thickness of the first metal layer is controlled to less than 1 micron.

14. The method of claim 11, wherein the thickness of the first metal layer is controlled to greater than 1 micron.

15. The method of claim 11, where in the first metal layer is silver.

16. The method of claim 11, wherein the first metal layer is a biocompatible metal.

17. The method of claim 11, wherein the first metal layer is gold or platinum.

18. A method of manufacturing a flexible fine stimulating lead, the method comprising the steps of:
providing an insulting biocompatible lead body,
affixing an electrode to the distal end of the lead body,
affixing a connector terminal to the proximal end of the lead body,
depositing at least one conductive metal layer on a non-conducting core to produce a fine wire lead for connecting the terminal to the electrode, and
depositing a biocompatible, insulting layer over the fine wire.

19. The method of claim 18, wherein the at least one conductive metal layer is from the group consisting of aluminum, titanium, platinum, gold, silver or combinations thereof.

20. The method of claim 18, wherein the non-conducting core is made of glass, or silica or combinations thereof.
